(19) United States
(12) Patent Application Publication
(10) Pub. No.: US 2017/0202687 A1
(43) Pub. Date: Jul. 20, 2017

(54) LINER

(71) Applicant: OTTO BOCK HEALTHCARE GMBH, Duderstadt (DE)

(72) Inventors: Klaus-Peter ANHALT, Rhumspringe (DE); Susann KIELE, Duderstadt (DE)

(73) Assignee: OTTO BOCK HEALTHCARE GMBH, Duderstadt (DE)

(21) Appl. No.: 15/326,823

(22) PCT Filed: Aug. 3, 2015
(86) PCT No.: PCT/EP2015/067800

§ 371 Date: Jan. 17, 2017

(30) Foreign Application Priority Data
Aug. 5, 2014 (DE) 10 2014 011 374.4

(51) Int. Cl. A61F 2/78

(52) U.S. Cl. CPC A61F 2/7812; A61F 2002/7818; A61F 2002/785; A61F 2002/802

(57) ABSTRACT

A liner for placing on the surface of the skin, comprising a base body made from a flexible, waterproof material having an outer side which is opposite the surface of the skin when in the applied state and an inner side which faces the surface of the skin when in the applied state, and channels which are open in the direction of the main surface and arranged on the inner side.

The invention relates to a liner for application to a surface of the skin, having a basic body produced from a flexible, watertight material and having an outside surface which, in the applied state, is remote from the surface of the skin and an inside surface which, in the applied state, faces the surface of the skin.

Liners from the prior art are provided for the purpose of being applied to a stump of a limb in order to produce an interface, in particular to a prosthesis. The liners can be realized as plastics material or silicone liners and, as a rule, comprise a closed cross section such that they surround the stump in an airtight manner. The inside surface can be provided with a coating which adheres to the surface of the skin. The stump, together with the liner, is introduced into a prosthesis socket and fixes the liner in the prosthesis socket by means of a locking device or by means of negative pressure. The generation of negative pressure within the framework of so-called suction socket liner technology is effected by the prosthesis socket being sealed in an airtight manner with respect to the prosthesis liner and the air present in the space between the prosthesis liner and the prosthesis socket being sucked or pressed out. The air is prevented from flowing back into the space by means of a non-return valve.

WO 2012/051385 A1 relates to a prosthesis liner which is realized as an interface between a stump and a prosthesis socket. The prosthesis liner comprises an elongated, substantially conical basic body in which a volume control pad is arranged. Circumferential sealing lips, which are directed radially outward, are arranged on the outside surface of the prosthesis liner in the distal region in order to provide improved sealing with respect to the inside surface of a prosthesis socket.

A similar embodiment of a liner is described in U.S. Pat. No. 8,052,760 B2 where sealing elements are arranged on the outer periphery of the prosthesis liner in order to ensure improved sealing of the space between the prosthesis liner and the prosthesis socket.

DE 101 53 796 A1 relates, among other things, to a liner to be used with a cup-shaped prosthesis socket which, at its distal end, comprises an opening through which a cord for pulling in the liner is run. The liner is locked in the prosthesis socket by means of a mechanical locking pin.

The problem in the case of prosthesis liners of this type is that the natural heat regulation mechanisms of a person, such as heat conduction, heat flow, heat radiation and evaporation, are only effective in a limited manner where there is full-surface direct skin contact with the liner surface. Heat flow in the form of convection cannot take place where there is full contact with the skin and sweat is only able to be evaporated in a limited manner as a result of the materials used.

U.S. Pat. No. 8,182,547 B2 consequently provides a multi-layered textile layer, the proximal edge ensuring airtight sealing. Air is able to be introduced into and sucked out of the space between the textile layer and the surface of the skin by means of ventilation channels in order to control both the negative pressure and the ventilation.

To remove sweat, U.S. Pat. No. 6,974,484 B2 provides for the insertion of an osmotic membrane which is covered by a liner. The osmotic membrane makes it possible to remove moisture from the stump, but blocks a return flow.

US 2012/0191218 A1 relates to a vacuum-supported carrier system for prosthesis devices on lower limbs, having an airtight socket with connections for a vacuum source and a liner which is sealed in an airtight manner at its edges and comprises porous regions between its edges. The liner is sealed with respect to the socket and an air-permeable distribution layer, for example produced from a textile, is arranged between the liner and the socket in order to distribute the vacuum applied to the socket. Air and moisture can be transported away from the surface of the skin and sucked out of the socket by means of a pump.

WO 2009/017762 A2 relates to a liner for use in prosthetic or orthotic devices. The liner comprises an inner layer with anti-slip components which form at least part of the periphery of the inner liner surface. The inner layer realizes a plurality of openings which lead to a porous element. As a result of the openings in the inner layer, the moisture is removed away from the stump and through the porous element to the surrounding area. The openings can be arranged in regions on the inside surface. Absorbent elements can be stored in the porous material. A closed outside layer can surround the perforated layer. A variant provides that the liner consists of two or more layers of interconnected polymer balls, the balls which abut against one another being connected together and being able to have the same diameter.

U.S. Pat. No. 8,282,686 B2 relates to a prosthesis socket arrangement for receiving an amputation stump, having a dimensionally stable outside shell which corresponds to the form of the stump to be received. An air-permeable inside layer is arranged on the inside surface of the outside shell at selected regions in order to be in contact with the surface of the skin and to distribute the applied vacuum. The inside layer is realized by a multi-layered textile. An inlet and an outlet, which is connected to a vacuum source, is provided on the outside shell.

It is the object of the present invention to provide a liner with which, on the one hand, good heat regulation and, on the other hand, a pleasant wearing experience is able to be achieved.

Said object is achieved according to the invention by a liner with the features of the main claim. Advantageous embodiments and developments of the invention are provided in the sub claims, the description and the figures.

The liner for application to a surface of the skin, having a basic body produced from a flexible, watertight material and having an outside surface which, in the applied state, is remote from the surface of the skin and an inside surface which, in the applied state, faces the surface of the skin, provides that channels which are open toward the surface of the skin are arranged on the inside surface. As a result, it is possible, on the one hand, for there to be contact between the inside surface of the liner and the surface of the skin and, on the other hand, equally for heat to flow in the form of convection and sweat to be evaporated. Consequently, direct contact is produced between the liner and both the surface of the skin and the inside surface of the prosthesis socket and, as a result, the prosthesis socket is coupled with the limb. Furthermore, heat and moisture is transferred directly along the surface of the skin through the and along the channels between the abutting surfaces, as a result of which the natural heat regulation mechanisms are maintained and convey a pleasant wearing feeling. The liner can be produced from watertight materials or from one single material which prevents moisture being removed from the surface of the skin through the material, materials being conditioned such that they prevent passage of water in liquid form. Elastomers, silicone and rubber-like substances or also closed-pore foams are suitable, in particular, as water-impermeable materials. The liners serve as an interface between the stump and a prosthesis socket. The surface of the skin is cooled efficiently as a result of the evaporation taking place on the surface of the skin, which increases the wearing comfort in a considerable manner.

The basic body can comprise a closed cross section with a proximal access opening, such that it is realized as a substantially conical hollow body. The distal end of the prosthesis liner is usually realized in a closed manner or comprises at least one opening for removing moisture. As an alternative to this, the basic body can comprise an opening at the distal end or, however, can be provided as an open cross section with closure elements, such that the initially planar prosthesis liner is placed around the stump and, by realizing a ring-shaped liner, can be fixed thereon by means of the closure elements.

In an advantageous manner, the basic body can comprise a closed outside surface in order to prevent moisture passing to the outside, such that no moisture is able to collect between the liner and the prosthesis socket. A textile layer, which conceals the outside surface of the liner completely or in part, can be attached or fastened on the outside surface of the liner. On account of the closed, watertight surface of the liner, the textile layer is kept away from the moisture located inside the liner, the liner acts as a moisture barrier between the surface of the skin and the outside surface of the liner or of the textile layer. The evaporation inside the liner in the channels or spaces brings about a cooling process without impairing the outside surface of the liner or allowing a substance to pass to the outside.

A coating, which is produced from a material that deviates from the material of the basic body, can be applied on the outside surface of the basic body. As a result, it is possible to produce multi-layered liners with different characteristics such that, for example, materials that are particularly well-tolerated by the skin and have good adhesive properties are able to be used, for example, on the inside surface, whilst particularly stable and abrasion-resistant materials are able to be used on the outside surface. The outer coating can also be a textile or comprise textile components. On the outside surface of the basic body a covering or an overlay can also be fitted in a reversible manner such that the liner can be formed as an inside liner with channels or pimples and an outside liner.

In an advantageous manner, the channels, which are open toward the surface of the skin, can be formed by nubs such that the channels are guided around the nubs. As a result, it is possible to provide a large surface along which the air is able to flow over the surface of the skin in order to achieve heat and moisture transfer. As a result of developing the channels by means of the nubs, it is possible to provide intersecting channels that are oriented in different directions, such that along with the usual flow direction, which follows the convection, it is possible to effect flow directions and air flows which extend transversely or at an angle thereto and, for example, follow a pump movement on account of the dynamic load when a prosthesis is in use. The ventilation and the heat and moisture transfer is further improved as a result of the channels intersecting or crossing one another. As an alternative to or in addition to the nubs, the channels can also be realized by webs which are arranged on the inside surface of the basic body. The nubs or webs can be realized integrally with the basic body, for example during the production process of the liner, or can be connected to the basic body subsequently as separate elements, for example as a result of subsequent adhesive bonding to or linking with the material of the basic body. To produce the nubs or webs, channels can be introduced into a substantially smooth-walled inside wall, for example milled or cut into said wall, as an alternative to this, the nubs or webs are introduced in the mold and are formed during the production process of the liner, in particular by molding. The nubs or webs are then an integral component part of the one-piece liner, as an alternative to this nubs or webs can be applied on the inside surface once a liner has been produced as the basic body. Nubs, in this case, are to be seen as elevations which stand out over a base area, the individual elevations being isolated from one another, as a result of which a structured surface with elevations and depressions is realized. The elevations and depressions can be distributed regularly or irregularly over the inside surface, it is also possible for the nubs to have forms and sizes that differ from one another in order to be able to make adjustments in line with local loads. Webs, compared to the nubs, comprise a larger longitudinal extension and can extend from one end of the liner to the other end.

The nubs and/or webs can be arranged in a regular arrangement on the inside surface, for example in different patterns, in order to be able to influence the heat regulation and removal of moisture. In particular, the nubs and/or webs can be arranged in a linear manner in the direction from the distal end to the proximal end in order to utilize the natural direction of flow of the heated air upward and to conduct away said air with the moisture contained therein as quickly as possible. It is also possible to arrange the nubs and/or webs such that they comprise channels, where applicable spiral-shaped channels, which extend in the circumferential direction, in order to direct the air flow. The nubs can be arranged offset to one another in the circumferential direction in order to be able to achieve flows in certain preferred directions.

The nubs and/or webs can be arranged over the entire inside surface in order to achieve as even as possible an abutment and pressure distribution on the stump surface. Along with the arrangement of patterns over the entire surface or the inside surface being completely covered with uniformly distributed nubs and/or webs, it is also possible and provided to have only partial covering with nubs and/or webs, such that only individual nub or web regions are arranged on the inside surface to be able to make natural heat regulation possible at particularly problematic places of the stump. The important point, in this case, is that both heated air and moisture are transferred via the spaces between the webs and/or nubs.

The nubs and/or webs can comprise an even surface which is directed to the surface of the skin, a slight curvature away from the surface of the skin also being possible in order to avoid areas of the skin being pinched under a compressive load. The surface of the nubs and/or webs can comprise a roughness in order to achieve improved comfort on account of the surface not being totally smooth.

The ratio of nub length to nub width can be between 0.8 and 2.5, thus ensuring the presence of channels or spaces that are large enough to remove the air and, apart from that, ensuring that the wearing feeling of the patients also remains pleasant.

The ratio of nub length to nub height can be between 1.5 and 2.5, thus ensuring that the nubs are not too tall and preventing the nubs from being pressed too far into the surface of the skin. Apart from that, the nub height is sufficiently tall to ensure an air flow in order to remove heat and moisture.

The distance between two nubs can be between half of the nub height and 1.5 times the nub height such that sufficient air circulation is present. The channels are then sufficiently large in order to enable the required removal of heat in the form of convection and sweat evaporation. The nubs and/or webs themselves can comprise a height of between 0.25 mm and 2 mm, in a preferred manner between 0.5 mm and 1 mm, in order to provide, on the one hand, sufficient height for air circulation and sweat removal and, on the other hand, sufficient stability against the liner outside surfaces folding back or slipping in relation to the surface of the skin. The web and/or nub height can be between 5% and 25% of the relative thickness of the wall of the liner, that is to say of the thickness of the wall of the liner at the base of the nub or the web.

The nub shape is preferred to be round, in particular oval or rectangular, as a result of which a realization of surface channels and a directed flow are able to be achieved.

In a preferred manner, the ratio of nub height to nub width or web height to web width is less than 1, such that the width of the nubs or webs is greater than the height thereof, thus preventing the channels located between the nubs and/or webs from being blocked as a result of the nubs and/or webs deforming when there is a large compressive load. The proposed ratio ensures that the spaces remain substantially continuous.

To support heat regulation, one or several connection devices can be provided for a ventilation device such that, for example, pumps or ventilators can be connected to the liner in order to suck out or push in air or also to remove moisture from the liner interior.

The channels realized in particular by the nubs can reach up to the proximal or distal edge of the prosthesis liner. They can be realized as ventilation or suction channels, it thus being possible for the surface of the skin which is not in direct contact with the nub surfaces or web surfaces or the channel intermediate material to be ventilated. In the case of air being supplied from outside, constant ventilation of the spaces or channels is ensured. The embodiment as suction channels provides that they are fluidically connected to a pump device, for example a suction pump which is driven in a mechanical or electric manner. It is equally possible to carry out ventilation when the channels communicate with a ventilation device, for example a pump that is connected on the pressure side. As a result of reversing the pump from suction mode to pressure mode, it is possible to utilize the channels and the pump for different types of operation, namely as active ventilation or active suction. If the channels extend up to the proximal and/or distal edge of the liner, the channels and consequently the space above the skin are ventilated and aerated via a differential pressure which prevails between the interior of the liner and the surrounding area. Where there is overpressure, air escapes out of the liner and where there is negative pressure, air flows into the liner, more precisely into the channels which are formed by the nubs. Both in the case of passive ventilation or aeration, operated with differential pressure, and active ventilation or aeration, brought about by means of a pump, the proximal edge of the liner can be realized in a closed manner when a supply of air is effected by means of ventilation or aeration openings, where applicable provided with a valve. The proximal edge of the liner can be sealed by the liner itself or with a sleeve such that the sweat vapor is removed in the distal end region, for example through a corresponding outlet opening, where applicable with a valve. Volume equalization in the case of a sealed edge takes place because of sweat production and phase change from liquid to gaseous during the evaporation. If all the moisture is not evaporated, the sweat can be removed in liquid form, which leads, thus, to a dry surface of the skin and improved hygiene. If negative pressure is to be applied to the liner, along with a separate pump this can also be effected by means of negative pressure that is present, where applicable, between the socket and the liner, by the socket interior being fluidically connected to the liner interior. A separate pump can be dispensed with in this way.

At least one connection for ventilation or aeration, which is fluidically connected to the channels, can be arranged in the liner in order to ensure that heat and moisture are removed from the liner or sufficient ventilation of the surface of the skin is carried out. The connection can be arranged at the distal end of the liner or at the proximal edge. The advantage of the arrangement of the connection on the distal end is that it lies, as a rule, at the lowest point of the liner during standing and walking, thereby producing, due to gravity, a direction of flow for moisture which can be utilized to remove the moisture. The connection can be coupled in a socket with a locking device for the liner and integrated in a so-called shuttle-lock.

A development of the invention provides that a phase change material is arranged on the liner or is integrated in the liner. By using a phase change material, also called a PCM, it is possible to exert a cooling effect on the air flow which is produced in the region of the channels or of the space between the skin and the liner. By means of the PCM, it is possible to provide active cooling for the user of the liner by exploiting the enthalpy of the thermodynamic state changes of a storage medium. Ventilation takes place in the channels between the surface of the skin and the liner such that the sweat evaporation contributes to the cooling. By means of the phase change material or the phase change materials, it is possible to achieve an enhanced cooling effect, for example by the air flow inside the liner at the same time being cooled by the phase change material.

It is possible for the channels to be in direct contact with the phase change material, for example by at least part of the channel walls consisting of a phase change material or being coated with the same such that the channels are directly fluidically connected to a phase change material. In addition, it is possible for a pump to be provided in order to convey the air flow. The flow through the channels can be effected by an applied negative pressure, as a result of which the evaporation of the body sweat is facilitated, which contributes to improved cooling. Insofar as cooling is not desired or reduced cooling is desired, it can be provided by means of a circuit or control means that the air exchange is reduced such that a reduction in or avoidance of heat transfer is brought about.

Exemplary embodiments of the invention are explained in more detail below by way of the accompanying figures, in which:

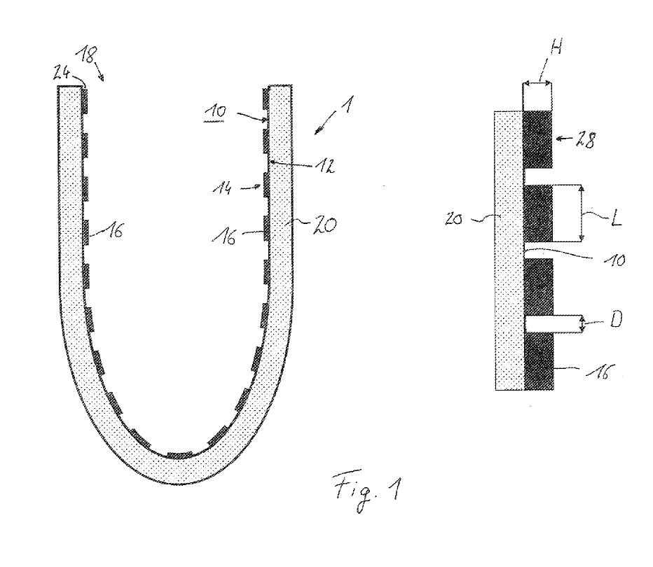

FIG. 1—shows a radial section through a liner and a representation of a detail;

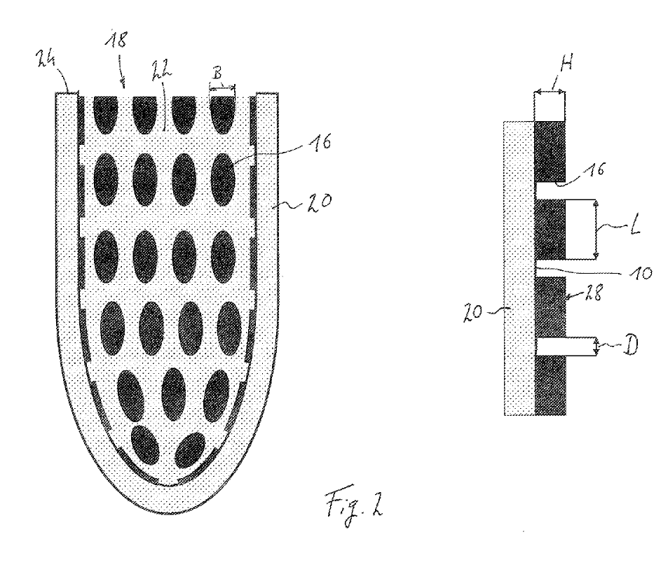

FIG. 2—shows a top view of a radial section according to FIG. 1;

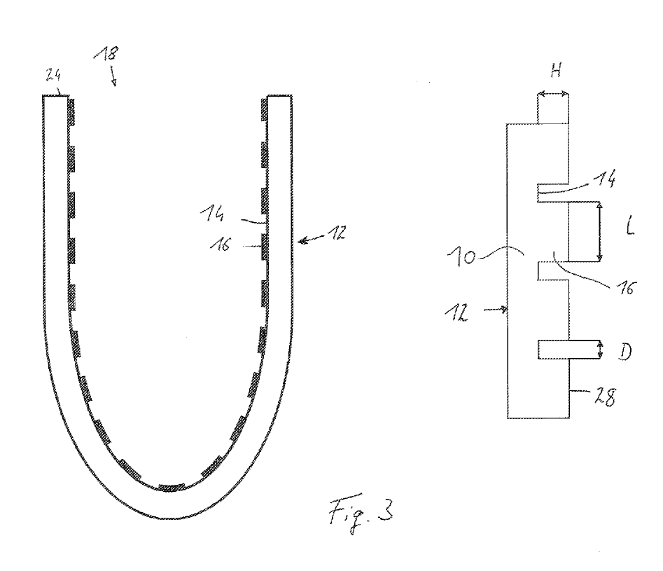

FIG. 3—shows a radial section through a one-piece liner with a representation of a detail;

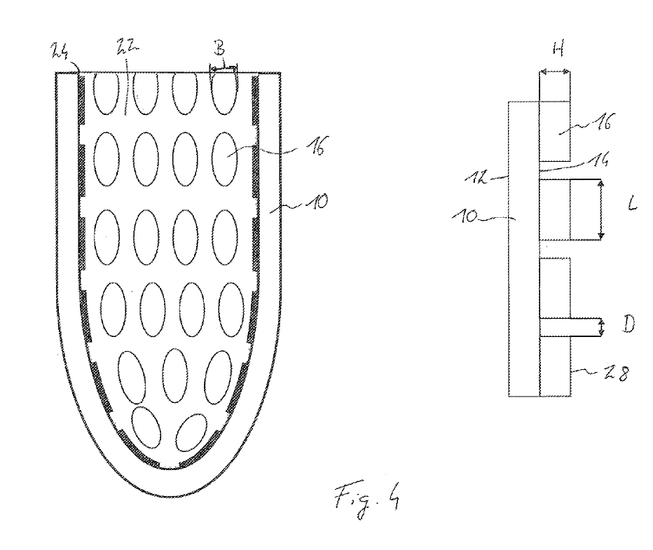

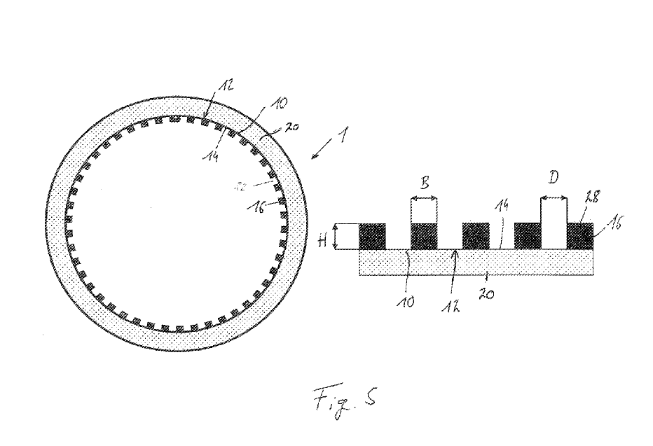

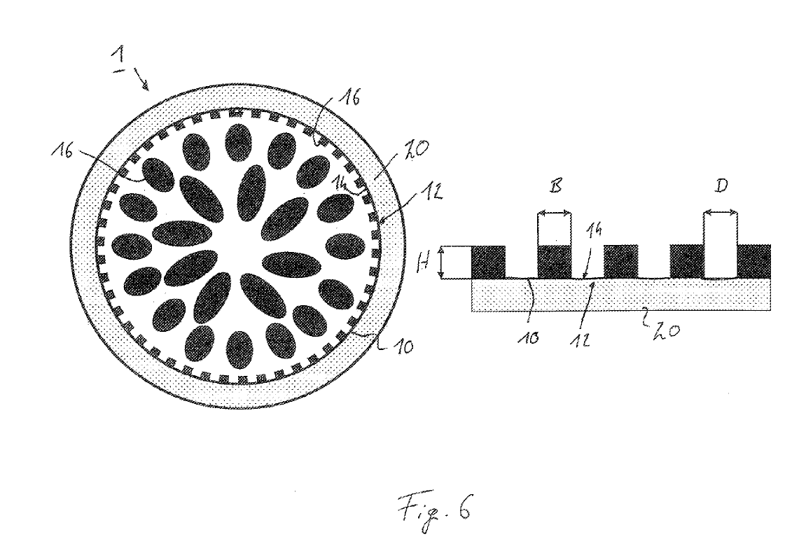

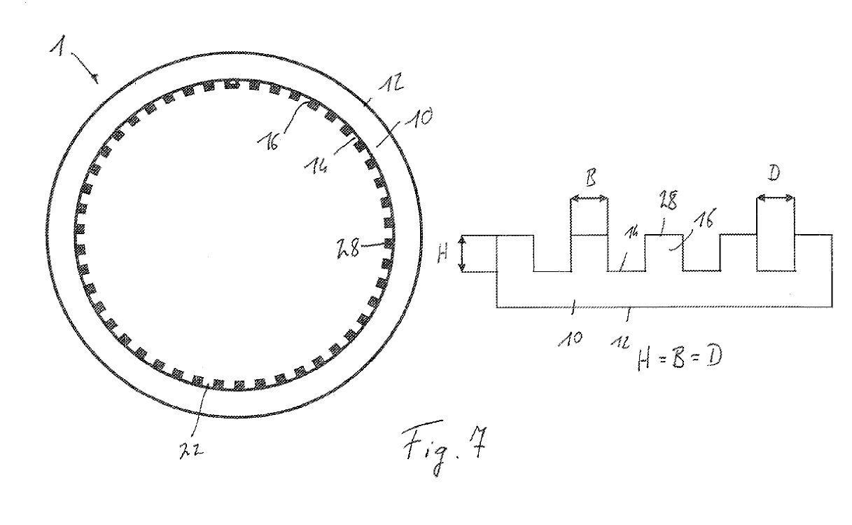

FIG. 4—shows a top view of a radial section and a representation of a detail with separate pimples;

FIG. 5—shows a cross sectional representation with a view of a detail;

FIG. 6—shows a top view of a cross sectional representation according to FIG. 5;

FIG. 7—shows a one-piece variant of a liner according to FIG. 5;

FIG. 8—shows a top view of a cross section of a liner with separate pimples;

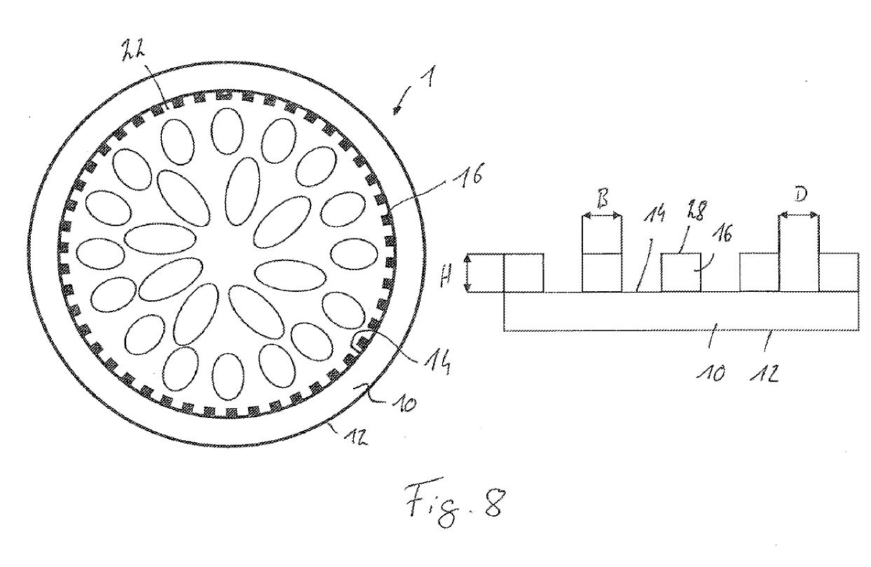

FIG. 9—shows a top view of two embodiments;

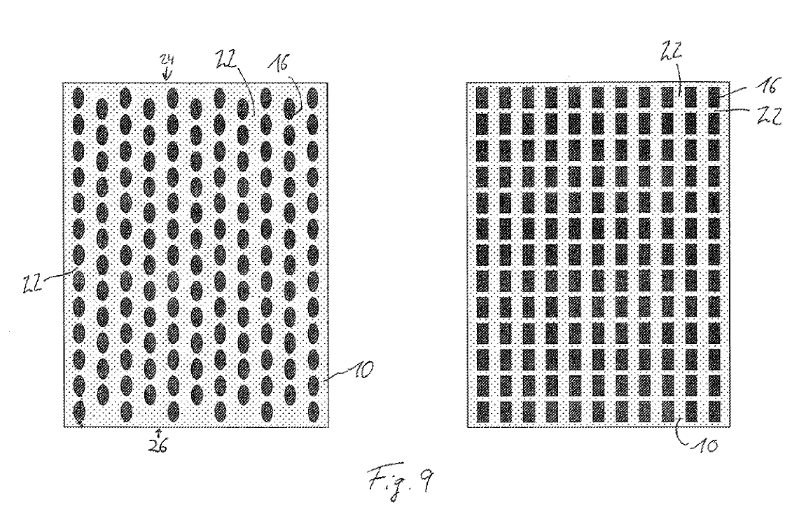

FIG. 10—shows a variant of FIG. 9;

FIG. 11—shows a radial section;

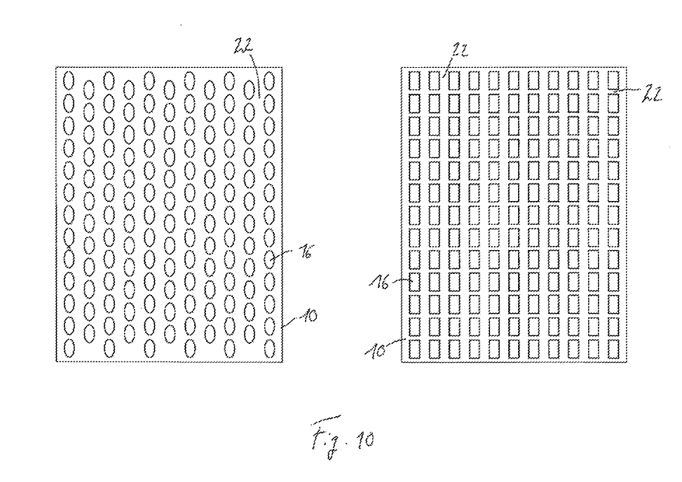

FIG. 12—shows a representation of a detail of FIG. 11;

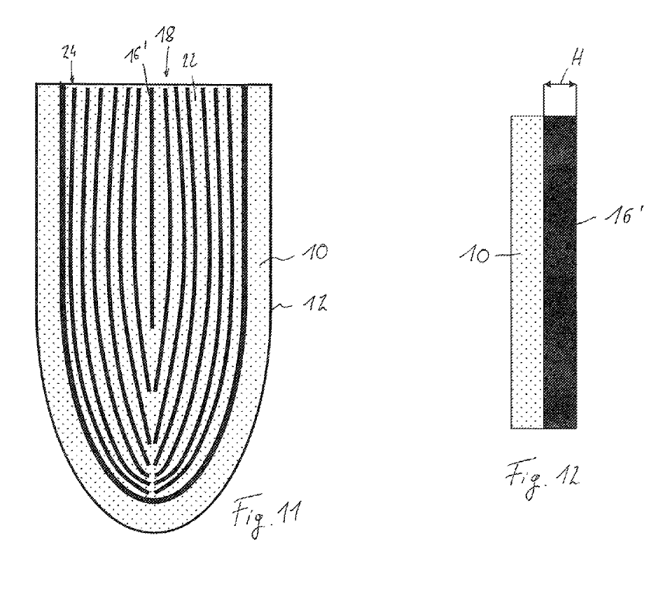

FIG. 13—shows a cross sectional view of FIG. 11;

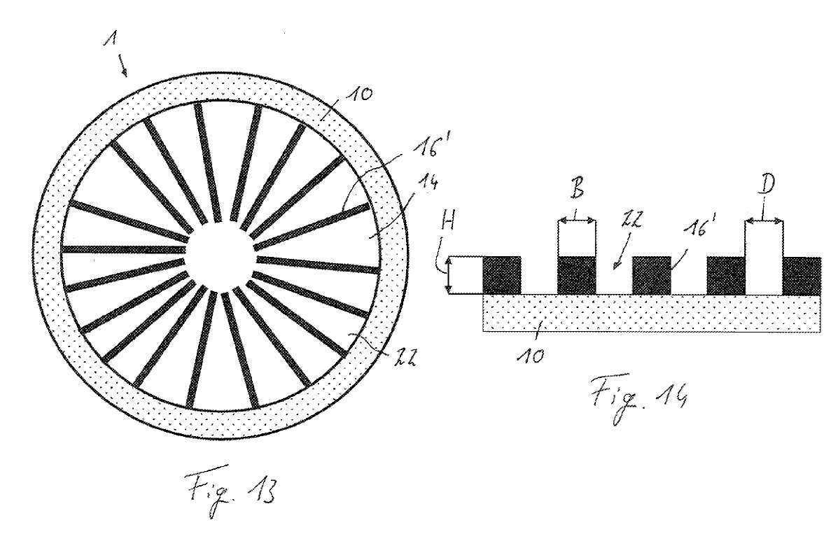

FIG. 14—shows a view of a detail of FIG. 13;

FIG. 15—shows a top view of a variant; and

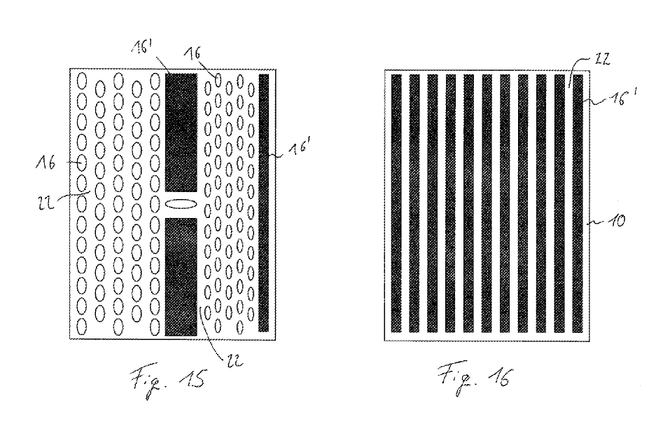

FIG. 16—shows a top view of a further variant.

In the left-hand representation, FIG. 1 shows a liner 1 with a radial section along the longitudinal extension in which only the cutting plane is shown. The liner 1 is realized as a closed liner and comprises a closed, distal end and an access opening 18, through which a stump (not shown) of a limb can be introduced into the liner 1. The liner 1 comprises a basic body 10 produced from a flexible, watertight and advantageously elastic material. The basic body 10 comprises an inside surface 14 which faces the stump in the applied state and an outside surface 12 which is remote from the surface of the skin. The outside surface 12 is realized in a closed manner such that no moisture from the stump is able to penetrate through the basic body 10 to the outside or to pass through the basic body 10 to the stump. Silicone or a plastics material, for example polyurethane, can be used as material for the basic body 10. A plurality of similar nubs 16, which project beyond the basic body 10 in the direction of the surface of the skin, are realized on the inside surface 14 of the basic body 10. A coating 20, which consists of a material which is different to the material of the basic body 10, is applied on the outside surface 12 of the basic body 10. As a result of the nubs 16, which comprise a planar surface 28 which faces the surface of the skin, ventilation of the surface of the skin is possible at those regions where the nubs 16 do not rest on the surface of the skin. As a result, it is possible to remove moisture and heat from the liner 1 such that hot air and moisture are able to emerge at the distal edge 24 of the liner.

An enlarged view of a detail of a liner wall with an outside coating 20, the basic body 10 and the nubs 16 which protrude therefrom and are directed to the inside surface, can be seen in the right-hand representation of FIG. 1. The nubs 16 comprise a planar surface 28 and a nub height H which is shorter than the nub length L. The ratio of nub length L to nub height H is between 1.5 and 2.5, it having been proved more advantageous when the nub length L is realized within a range of between 0.7 mm and 1.2 mm. The distances D between two nubs 16 are advantageously shorter than the nub height H and in the exemplary embodiment shown are approximately half the nub height. It can also be that the distances are chosen to be longer such that the distance D between two nubs 16 can be up to 1.5 times the nub height H. The distance can be between 0.2 mm and 1.2 mm.

FIG. 2 shows an alternative representation of the liner 1 according to FIG. 1. In the left-hand representation, it can be seen that the form of the nubs 16 is oval, the distance D is the respectively shortest distance between two nubs 16, the nub length L, in this case, designates the longer longitudinal extension and the nub width B the shorter extent of the nubs 16. The ratio between nub length and nub width can be between 1 and 2.5. The arrangement of the nubs 16 extends substantially uniformly over the entire inside surface 14 of the basic body 10, realizing intermittent channels 22 which are open inwardly and extend initially substantially from distal to proximal and at the same time, on account of the arrangement of nubs 16, realize channels which run around at the same height and intersect the channels 22 in the proximal-distal direction such that free passage of heat and moisture to the proximal edge 22 is possible from all body part that are not covered by nub surfaces 28. The emission of heat and moisture is effected, in this case, in the space between the basic body 10 and the surface of the skin.

It can be seen in the right-hand representation that the coating 22 consists of a material that differs from the material of the basic body 10, the basic body 10 and the nubs 16 are realized in one-piece or integrally and comprise good adhesion when applied on the skin. The outside coating 20 can be chosen from a material which comprises particularly good mechanical properties and good long-term durability, which does not necessarily have to concur with what the skin will tolerate, such that there is a large degree of freedom for the design of the liner 1 as a result of being able to use two different materials.

FIG. 3 shows a radial section analogous to FIG. 1, it is clear in the right-hand representation that the entire liner 1 is produced from one single material and the nubs 16 are formed integrally on the inside surface 14 of the basic body 10.

A variant to FIG. 3 is shown in FIG. 4 where the nub shape corresponds to that of FIG. 2, the nubs 16 and the basic body 10, however, are not formed in one piece, but have been subsequently attached to the basic body 10, for example as a result of adhesive bonding or by subsequent connection and a substance-to-substance bond. The liner 1 according to FIG. 4 provides for the use of only one material. As a result of attaching the nubs 16 subsequently, a greater range of variation can be covered when manufacturing liners as consideration can be shown for individual characteristics.

FIG. 5 shows a cross section through a liner 1 according to FIG. 1 perpendicular to the longitudinal extension. The channels 22 are produced as a result of arranging the nubs 16 in a uniform manner on the inside surface 14 of the basic body 10. FIG. 5 shows the closed cross section of the liner 1 such that the liner 1 is able to surround the stump (not shown) completely. In the exemplary embodiment according to FIG. 5, the nub height H is equal to the nub width B is equal to the distance D between two nubs 16 and consequently also to the width of the channels 22.

FIG. 6 shows an alternative representation of the liner 1 according to FIG. 6 in which the arrangement of the nubs 16 can be seen better. The nubs 16 extend along the substantially cylindrical side wall of the basic body 10 as well as in the conically tapering distal end region, no nubs 16 being provided at the lowest point of the liner.

FIG. 7 shows a cross sectional representation of a liner according to FIG. 3 and FIG. 8 shows a cross section through a liner according to FIG. 4. The difference between the embodiments according to FIGS. 7 and 8 is that the basic body 10 does not comprise a coating on the outside surface 12. The material of the nubs 16 in the exemplary embodiment in FIGS. 4 and 8 can also deviate from the material of the basic body 10.

FIG. 9 shows a top view of the inside surface 14 of a basic body 10 with different nub forms. The left-hand representation provides oval nubs 16 and the right-hand representation rectangular nubs 16. In the longitudinal extension from the distal edge 26 to the proximal edge 24, the nubs 16 extend substantially parallel and in the left-hand representation the nubs are vertically offset such that diagonally-extending channels 22 are realized next to the parallel-extending channels 22, in the right-hand representation the nubs 16 are distributed uniformly on the basic body 10 and, in each case, run parallel to one another. The basic body 10 can be provided with closure or fastening elements (not shown) such that it can be wound around a stump or a limb and fixed to it such that a liner which is open at the distal edge 26 and at the proximal edge 24 is created which can serve as an interface for a prosthetic or orthotic fitting. In principle, it is also possible to provide a sleeve-shaped or hose-shaped liner with an open distal end.

The nubs in the exemplary embodiment according to FIG. 9 can be realized from a material that differs to the basic body 10. FIG. 10 shows the top view of the basic body according to FIG. 9 with nubs 16 produced from the same material as the basic body 10. The nubs 16 according to FIG. 10 can be molded at the same time as the process for forming the basic body 10 or can be attached subsequently. The nubs 16 on both basic bodies 10 form a structured surface with alternating elevations and depressions such that air circulation and moisture removal is able to be effected in the spaces between nubs.

FIG. 11 shows a radial section of the liner 1 with a basic body 10 and an outside surface 12. The insertion opening 18 on the distal edge 24 enables handling and access into the liner 1. Webs 16′ which extend radially inward are provided on the inside surface of the basic body 12 such that channels 22, which extend from the closed distal end to the open proximal edge 24, are realized between the webs 16′. The channels 22 are oriented in a substantially straight manner on the inside surface of the basic body 10 and enable direct removal of sweat and exchange of air. The basic body 10 is realized so as to be closed-walled and substantially watertight.

FIG. 12 shows a representation of a detail of the liner 1 with the basic body 10 and the radially inwardly projecting web 16′. The height H of the web 16′ is dimensioned such that, on the one hand, there is a sufficiently large amount of stability and, on the other hand, a sufficient distance between the inside surface of the basic body 10 and the surface of the skin (not shown) in order to enable removal of air and moisture.

FIG. 13 shows a cross section through the liner 11 and it can be seen from the figure that the height H and the width B of the webs 16′ correspond to one another in a substantial manner such that a square cross section, proceeding from the basic body 10, is produced for the web 16′. The distance D between two webs 16′ corresponds substantially to the width B of the web 16′, a possible range of dimension for the height H, the width B and the distance D is between 0.3 and 0.7 mm, but is not restricted to this.

FIG. 15 shows a top view of a first variant of the inside surface of a liner where, along with nubs 16 in different sizes and orientations in an oval form, webs 16′ are attached or realized on the basic body 10. The channels 22 are realized between the webs 16′ and nubs 16. It can be seen in FIG. 15 that the webs 16′ do not have to run continuously from the distal to the proximal end, rather interruptions are also possible. In the exemplary embodiment shown, one nub 16 is arranged between two web portions 16′. In principle, it is possible to arrange nubs 16 and webs 16′ of different sizes and orientations on the inside surface of the basic body 10.

A further variant is shown in FIG. 16 where a plurality of webs 16′ are arranged uniformly spaced apart from one another on the inside surface of the basic body 10. Uniformly realized channels 22, which extend in a straight manner, are correspondingly realized between the webs 16′.

1. A liner for application to a surface of the skin, the liner comprising:
a basic body produced from a flexible, watertight material and comprising:
an outside surface which, in an applied state of the liner, is remote from the surface of the skin;
an inside surface which, in the applied state, faces the surface of the skin;
channels which are open toward the surface of the skin and arranged on the inside surface.

2. The liner as claimed in claim 1, wherein the basic body comprises a closed cross section with a proximal access opening.

3. The liner as claimed in claim 1, wherein the basic body comprises a closed outside surface.

4. The liner as claimed in claim 1, further comprising a coating, which is produced from a material that deviates from the material of the basic body, applied on the outside surface.

5. The liner as claimed in claim 1, wherein the channels are formed by at least one of nubs and webs.

6. The liner as claimed in claim 5, wherein the at least one of nubs and webs are arranged in a regular arrangement on the inside surface.

7. The liner as claimed in claim 5, wherein the at least one of nubs and webs are arranged over the entire inside surface.

8. The liner as claimed in claim 5, wherein the at least one of nubs and webs comprise an even surface which is directed to the surface of the skin.

9. The liner as claimed in claim 5, wherein when the channels are formed by nubs, a ratio of nub length to nub width is between 0.8 and 2.5.

10. The liner as claimed in claim 5, wherein when the channels are formed by nubs, a ratio of nub length to nub height is between 1.5 and 2.5.

11. The liner as claimed in claim 5, wherein a distance between two nubs or between two webs is between half of a height of the nubs or webs and 1.5 times the height of the nub or web.

12. The liner as claimed in claim 5, wherein a ratio of a height of the numbs or webs to a width of the nubs or webs is less than 1.

13. The liner as claimed in claim 1, wherein the channels at least one of reach up to a proximal or distal edge of the liner or are formed as suction or ventilation channels.

14. The liner as claimed in claim 1, further comprising at least one connection device provided for a ventilation device.

15. The liner as claimed in claim 1, further comprising a connection for ventilation or aeration arranged in the liner, the connection being fluidically connected to the channels.

16. The liner as claimed in claim 1, further comprising a phase change material arranged on the liner or integrated in the liner.

17. The liner as claimed in claim 16, wherein the channels are fluidically connected to the phase change material.

18. A prosthetic liner configured to mount to residual limb, comprising:
a basic body, comprising:
a flexible, watertight material;
an outside surface configured to face away from the residual limb;
an inside surface configured to face the residual limb;
channels positioned on the inside surface and configured to open toward the residual limb.

19. The liner as claimed in claim 18, wherein the basic body comprises a closed cross section with a closed distal end and a proximal access opening.

20. The liner as claimed in claim 18, further comprising a coating positioned on the outside surface, the coating comprising a material that is different from the material of the basic body
